(19) United States
(12) Patent Application Publication
(10) Pub. No.: US 2008/0221479 A1
(43) Pub. Date: Sep. 11, 2008

(54) Integrated Imaging and Biopsy System with Integrated Utilities

(76) Inventors: Paul G. Ritchie, Loveland, OH (US); Trevor W. V. Speeg, Williamsburg, OH (US); Timothy G. Dietz, Terrace Park, OH (US); John A. Hibner, Mason, OH (US)

Correspondence Address: FROST BROWN TODD, LLC, 2200 PNC CENTER, 201 E. FIFTH STREET, CINCINNATI, OH, 45202, US

(21) Appl. No.: 11/852,750

(22) Filed: Sep. 10, 2007

Related U.S. Application Data
(60) Provisional application No. 60/893,454, filed on Mar. 7, 2007.

(51) Int. Cl. A61B 10/02

(52) U.S. Cl. 600/563; 600/562

(57) ABSTRACT

A diagnostic station integrates patient support, imaging, biopsy, and treatment. An illustrative version of a prone mammography table localizes a breast with an imaging modality (e.g., X-ray, etc.) based upon a rotating C-arm that may encircle the localized breast. A biopsy system is integrated into the controls and displays or user interface of the diagnostic station, sharing integrated utilities (e.g., vacuum, power, data communication, etc.). Ancillary devices may be identified and authenticated by the integrated system, such as to base available functionality on the identification and/or authentication of an ancillary device. Ancillary devices that may be integrated with the system may include devices that are operable to perform surgical, therapeutic, diagnostic, or other functions.

### PRIORITY

This application claims priority to U.S. Provisional Application Ser. No. 60/893,454, entitled “Integrated Imaging and Biopsy System,” filed Mar. 7, 2007, the disclosure of which is incorporated by reference herein.

### BACKGROUND

Embodiments of the present invention are related to an apparatus for medical examination and/or treatment. More specifically, embodiments of the present invention relate to an apparatus and method for imaging a female breast and guiding a biopsy and/or treatment procedure, and more particularly as part of an integrated system.

Diagnostic apparatuses that image the body to provide diagnostic information, localization, and therapeutic targeting are well known in the art. These diagnostic devices may utilize X rays, PET (Positive Emission Tomography), Magnetic resonance, ultrasound, or other energy technology. Such devices may have some interface with the human body, whether they are positioning/clamping devices such as may be used with X-ray tables, or transducers such as an ultrasound transducer. They may also involve requiring the patient to lie or stand in a variety of positions, either to provide access for the physician, or to fix or locate body tissue in relation to the device. For the purposes of this disclosure, all such diagnostic apparatus will be referred to as a diagnostic table, without limitation to such devices that require the patient to lie on a surface.

In the prior art, one example of a prone stereotactic (X-ray) device that may be used for localization of breast abnormalities is illustrated by U.S. Pat. No. 5,289,520, the disclosure of which is incorporated by reference herein for purposes of illustration only. A mammography system is illustrated by U.S. Pat. No. 6,545,280, the disclosure of which is incorporated by reference herein for purposes of illustration only. U.S. Pat. No. 6,678,546 describes how a diagnostic table can be used, the disclosure of which is incorporated by reference herein for purposes of illustration only. In addition, devices that biopsy, or physically remove tissue either for diagnosis or for cosmetic or therapeutic purposes, are known in the art. For instance, U.S. Pat. No. 5,526,822, entitled “Method and Apparatus for Automated Biopsy and Collection of Soft Tissue,” describes such a system, and is incorporated by reference herein. Such systems can be externally affixed to diagnostic tables to allow a biopsy needle or device to be guided by the diagnostic table, such as is described by U.S. Pub. No. 2004/0230133.

While a variety of diagnostic tables and biopsy devices have been made and used, it is believed that no one prior to the inventor has made or used a device or system as described in the appended claims.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 is a perspective view of a mobile medical vehicle cut away to show a patient support mammography table with an integrated imaging and biopsy system to form a diagnostic station.

FIG. 2 is a perspective view of a biopsy probe assembly of the integrated imaging and biopsy system of FIG. 1.

FIG. 3 is a control module and the biopsy probe system of FIG. 2 for use with the diagnostic station of FIG. 1.

FIG. 4 is a perspective view of a biopsy probe assembly of the integrated imaging and biopsy system of FIG. 1.

FIG. 5 is a top perspective view of the prone patient supporting the mammography table of FIG. 1.

FIG. 6 is a left side perspective view in elevation of the same table showing the base, pedestal and angularly movable C-arm carrying the X-ray tube and the image receptor as well as the separate compression arm carrying compression plates and needle guide omitted for clarity.

FIG. 7 is a front elevation view of the patient support mammography table of FIG. 1.

FIG. 8 is a functional block diagram of the integrated imaging and biopsy system of FIG. 1.

FIG. 9 is a block schematic showing various components in communication with an integrated control system.

FIG. 10 is an exemplary graphical user interface that may be used with the integrated control system of FIG. 9.

FIG. 11 is a flow chart showing an exemplary identification and authentication workflow.

FIG. 12 is a flow chart showing an exemplary authentication workflow.

FIG. 13 is a partial view of a biopsy probe and a table arm with exemplary complimentary features.

FIG. 14 is a block schematic showing various utilities that may be integrated with a diagnostic station.

FIG. 15 is a schematic showing an exemplary harmonic device integrated with a table.

FIG. 15 is a schematic showing an exemplary brachytherapy device integrated with a table.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

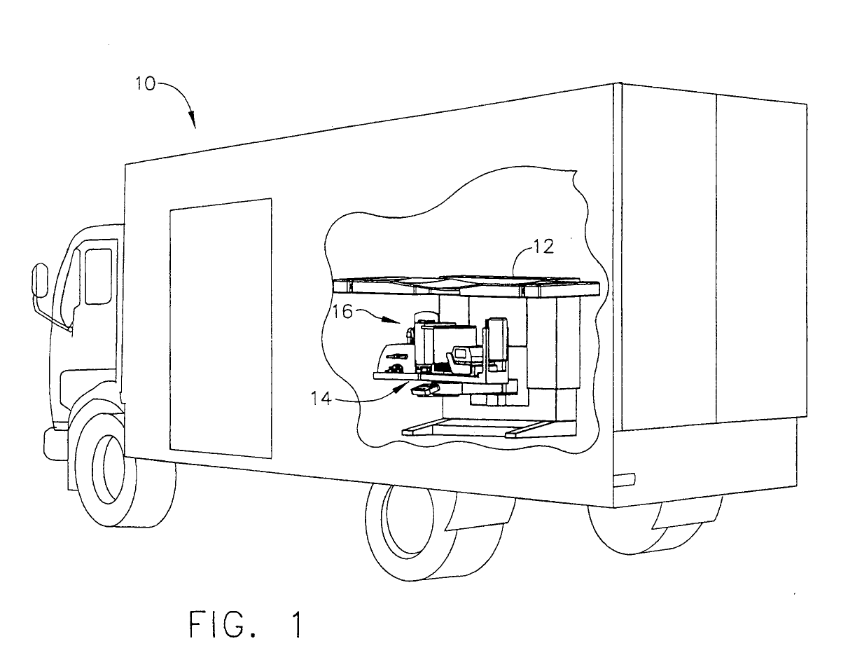

Turning to the Figures, wherein like numerals denote like components throughout the several views, in FIG. 1, a mobile medical vehicle 10 includes a patient support table 12 having an integrated imaging, biopsy and treatment system 14 forming a diagnostic station 16, which in the illustrative depiction is configured to support a prone patient, though a diagnostic station 16 may alternatively be configured to support a patient in a variety of other positions. Other embodiments may include a diagnostic station 16 in a non-mobile station, such as in a hospital, clinic, or elsewhere.

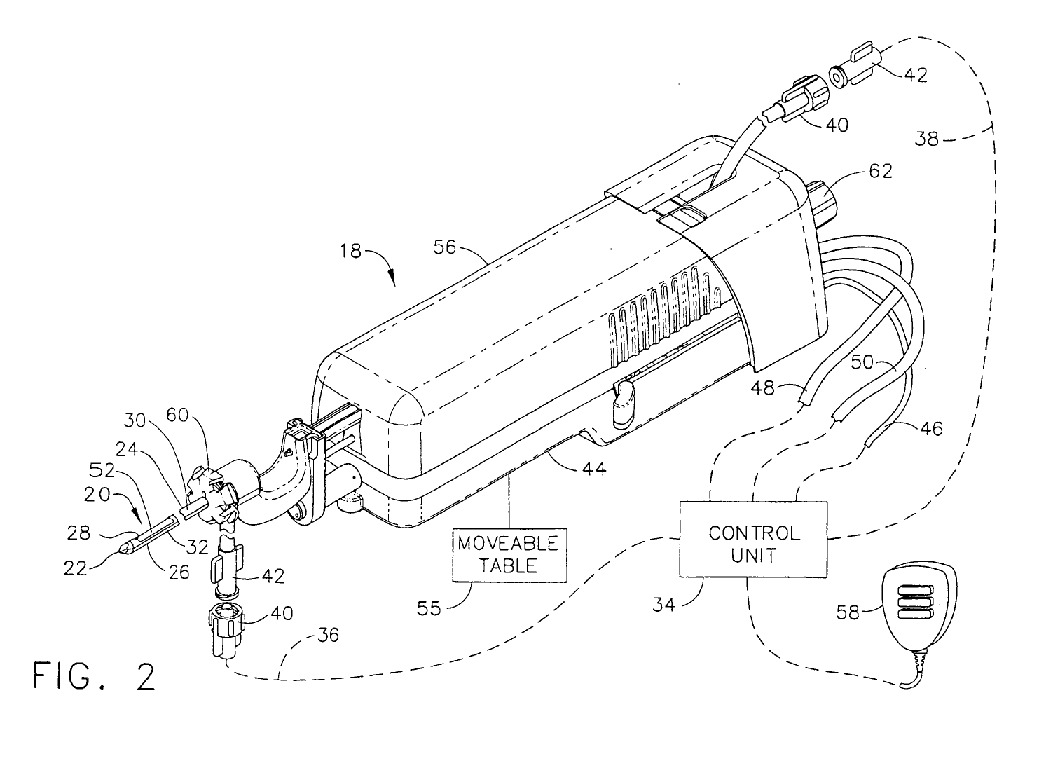

In FIG. 2, an illustrative biopsy probe assembly 18 for use with the diagnostic station 16 of the present example includes an elongated piercer 20 having a piercer tip 22 for penetrating soft tissue of a surgical patent. Piercer 20 comprises a piercer tube 24 and a vacuum chamber tube 26. On the side of the distal end of piercer tube 24 is a port (side aperture) 28 for receiving tissue to be extracted from the surgical patient proximally through a piercer lumen 30 defined by the piercer tube 24. The elongated vacuum chamber tube 26, which is joined along its length to the piercer tube 24, defines a vacuum lumen 32. Piercer lumen 30 is in fluid connection with vacuum lumen 32 via a plurality of vacuum holes (not shown) located in the bottom of the “bowl” defined by port 28. Vacuum holes are small enough to remove the fluids but not large enough to allow excised tissue portions to be removed through lateral vacuum lumen 32. The metallic or ceramic sharp piercer tip 22 is fixedly attached to the distal end of piercer 20. It is designed to penetrate soft tissue, such as the breast tissue of a female surgical patient. One example is a piercer tip 22 having a three sided, pyramidal shaped point, although the tip configuration may also have other shapes. Of course, those of ordinary skill in the art will immediately recognize that biopsy probe assembly 18 as described herein is merely exemplary. A variety of other biopsy probe assemblies having any other suitable configuration or components may be used. Furthermore, a variety of alternative devices (e.g., those that perform operations other that obtaining biopsies) may be used within the system of the present example.

The biopsy probe assembly 18 of the present example may be fluidly, mechanically, and/or electrically connected to a biopsy control unit 34. It should be appreciated from the description that follows that the biopsy control unit 34 may comprise a legacy dedicated controller or may be in communication with the diagnostic table 16 or an integrated function provided by the diagnostic station 16. Axial vacuum to the probe assembly 18 may be obtained by fluid connection to the biopsy control unit 34 by detachable fluid connection of a lateral vacuum line 36 that communicates proximally with the vacuum lumen 32 and an axial vacuum line 38 that communicates proximally with the piercer lumen 30 to biopsy control unit 34. Lateral vacuum line 36 and axial vacuum line 38 may be made from a flexible, transparent or translucent material, such as silicone tubing, allowing for visualization of the material flowing through them. In the present example, lateral connector 40 and axial connector 42 are female and male luer connectors, respectively, commonly known and used in the medical industry. To avoid reversed installation, the vacuum lumen is connected to male luer connector 42 that engages a female luer connector 40 of the lateral vacuum line 36, with reversed connectors 40, 42 for the piercer lumen 30.

In the present example, base 44 is operatively connected to biopsy control unit 34 by a control cord 46, translation shaft 48, and rotation shaft 50 for independently longitudinally translating and/or rotating, respectively, a cutter tube 52 that translates within the piercer lumen 30 to sever tissue prolapsed into the port 28 under the urging of vacuum supplied through the piercer and vacuum lumens 30, 32. The cutter tube 52 is visible in FIG. 2 within the port 28, having been distally positioned. Translation shaft 48 and rotation shaft 50 may be flexible so as to permit for the ease of mounting of the base 44 of the biopsy probe assembly 18 to a movable table 55 of the patient supporting mammography table 12. An upper portion 56 of the biopsy probe assembly 18 is engaged to the base 44. It should be appreciated that one shaft may be employed through a transmission into the respective translation and rotation motions, especially at a fixed ratio. It will also be appreciated that rotation and/or translation of cutter tube 52 may be provided in a variety of alternative ways, in addition to or in lieu of translation shaft 48 and/or rotation shaft 50. By way of example only, rotation and/or translation of cutter tube 52 may be provided by a pneumatic motor and/or piston assembly in communication with a pressurized fluid supply. As another merely illustrative alternative, one or more motors may be provided within biopsy probe assembly 18 to provide rotation and/or translation of cutter tube 52. Still other ways in which a cutter tube 52 may be rotated and/or translated will be apparent to those of ordinary skill in the art.

A tethered remote control 58 may be operatively and removably connected to biopsy control unit 34. Remote control 58 may be used by the surgical biopsy system operator to control the sequence of actions performed by the biopsy assembly 18. In the present example, a front thumbwheel 60 attached to the piercer 20 allows rotation of the piercer 20, relative to upper portion 56 of the biopsy probe assembly 18, to orient the port 28. An aft rotation knob 62 may also be turned to effect rotation of the piercer 20. Alternatively, piercer 20 may be configured such that it does not rotate, such that it rotates with assistance from a motor (e.g., electrical or pneumatic, etc.) or from some other device, or in any other suitable fashion.

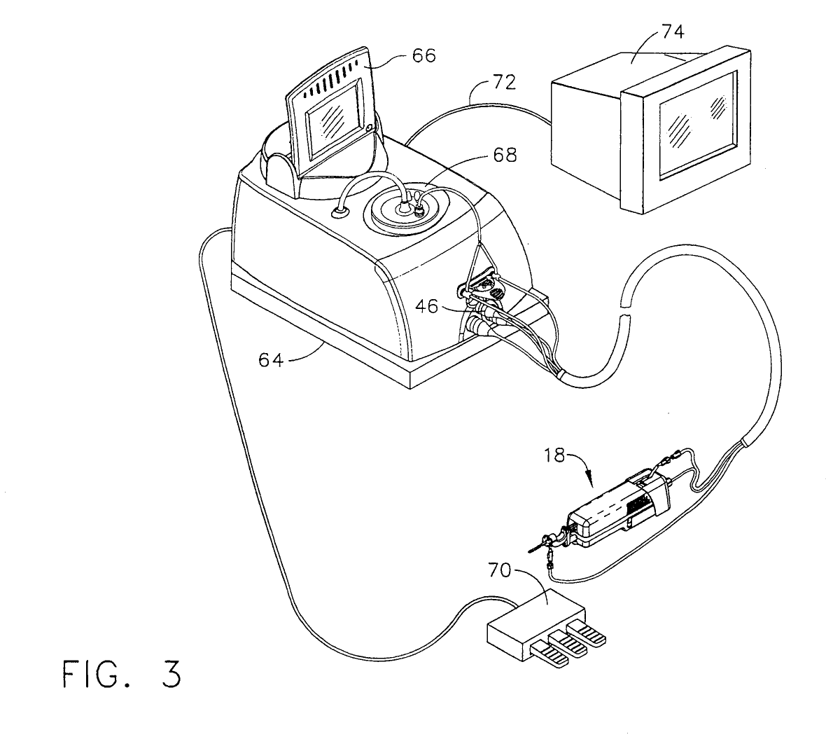

As will be appreciated with the benefit of the present disclosure, varying degrees of synergy and/or independence may be represented in the degree of integration of the biopsy system 20 into the diagnostic table 16 of the present example. In FIG. 3, a control unit 34 is incorporated in a control module 64 that includes a dedicated biopsy display 66, a vacuum generation and fluid separation apparatus 68, and shaft actuation and control sensing (not shown) via the control cord 46 to the biopsy probe assembly 18. Instead of or in addition to the hand operated remote 58 (FIG. 2), a tethered foot operated device 70 may be used that communicates with the control module 64. A communication link 72 is depicted going to a display monitor 74 from the control module 64 indicating a repeated display or inclusion of biopsy related data into another display of other data (e.g., imaging data, etc.). It will be appreciated that communication link 72 may be wired (e.g., USB, Ethernet, serial connector, etc.) or wireless (e.g., RF, Bluetooth, etc.).

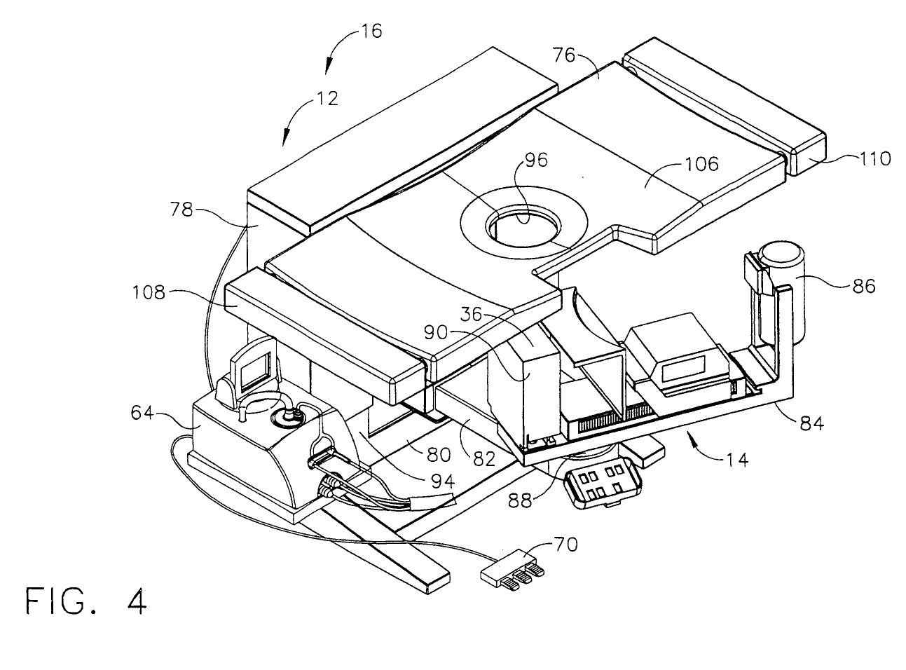

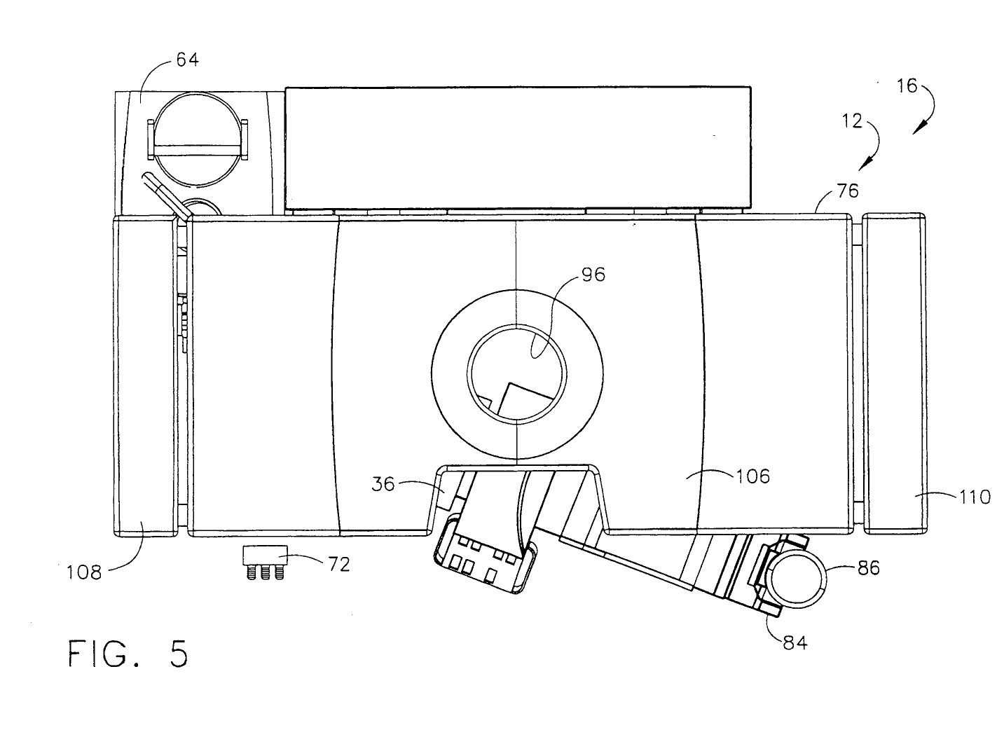

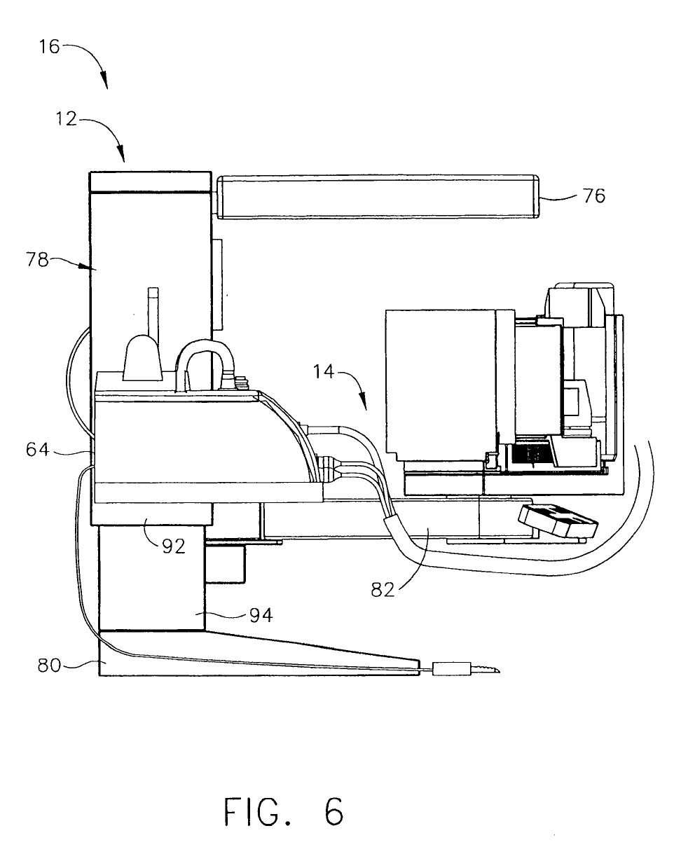

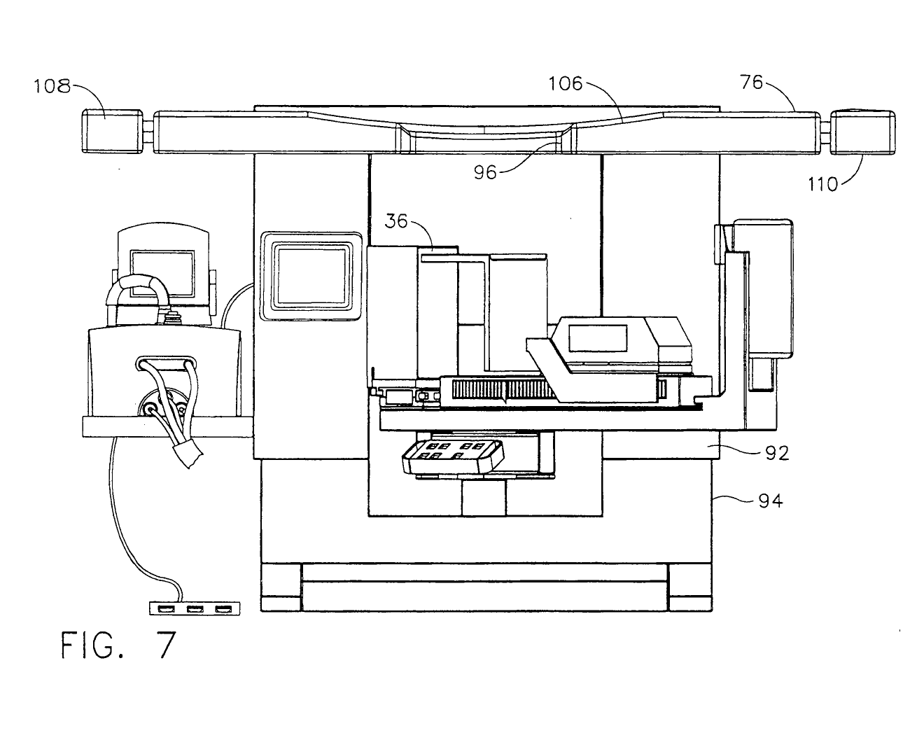

In FIGS. 4-7, the patient support, breast localization, and imaging components of the patient support mammography table 12 are substantially as described in U.S. Pat. No. 5,289,520, the disclosure of which is hereby incorporated by reference in its entirety. Of course, any other type of table 12 having any other suitable components or configuration may be used as an alternative to the one depicted in FIGS. 4-7. As shown, patient supporting mammography table 12 comprises a platform 76 on which the patient rests in a prone position, supported by a rear pedestal 78 upstanding from the rear portion of a table base 80, all as shown in FIGS. 4, 6. Pedestal 78 in this example incorporates table elevating means to raise and lower the table 12 within limits for convenience of the patient and attending personnel. In other embodiments, the elevational position of pedestal 78 is substantially fixed and table 12 cannot be raised or lowered.

Protruding forward over the lower part of base 80 from the front face of pedestal 78 is a ledge 82 sturdily constructed to provide underlying support for an angularly movable “C-arm” 84. Arm 84 is shaped like a letter “C” lying on its back, with one upstanding end mounting the X-ray source or mammography unit tube head 86. A pivot axis 88, about which C-arm 84 is mounted for angular rotation relative to ledge 82, is close to the opposite upstanding end of the C-arm 84, and this upstanding end incorporates a charge coupled device (CCD) sensor folded optical system 90 enclosed in a light-tight housing. Other components may be provided on or in C-arm 84. Similarly, C-arm 84 may be substituted or supplemented with other components, or may be simply omitted altogether.

An upper portion 92 of pedestal 78, supporting the table platform 76 at its upper end and the ledge 82 at its lower end, is capable of vertical downward movement from the raised position to a lowered position in which the ledge 82 is close to base 80. This vertical adjustment motion is provided by telescoping upper pedestal portion 92 over an underlying lower pedestal portion 94. Further adjustability of the system may be provided by separate vertical adjustment of ledge 82 relative to upper portion 92 of the pedestal 78. Other ways in which adjustability may be provided, to the extent that it is provided at all, will be apparent to those of ordinary skill in the art.

As shown in FIGS. 4, 5 and 7, a central aperture 96 is provided in the central portion of platform 76 accommodating one or both of the female patient's breasts hanging pendulently therethrough as the patient lies face down on platform 76. A relatively thin image receptor 98 is positioned close to the pivot axis 88 about which the C-arm 84 moves angularly, and the pivoting movement of C-arm 84 about axis 88 allows the image receptor 98 to be positioned between the patient's breasts, or against the underside of either breast, by making minor adjustments in the position of axis 88 relative to ledge 82.

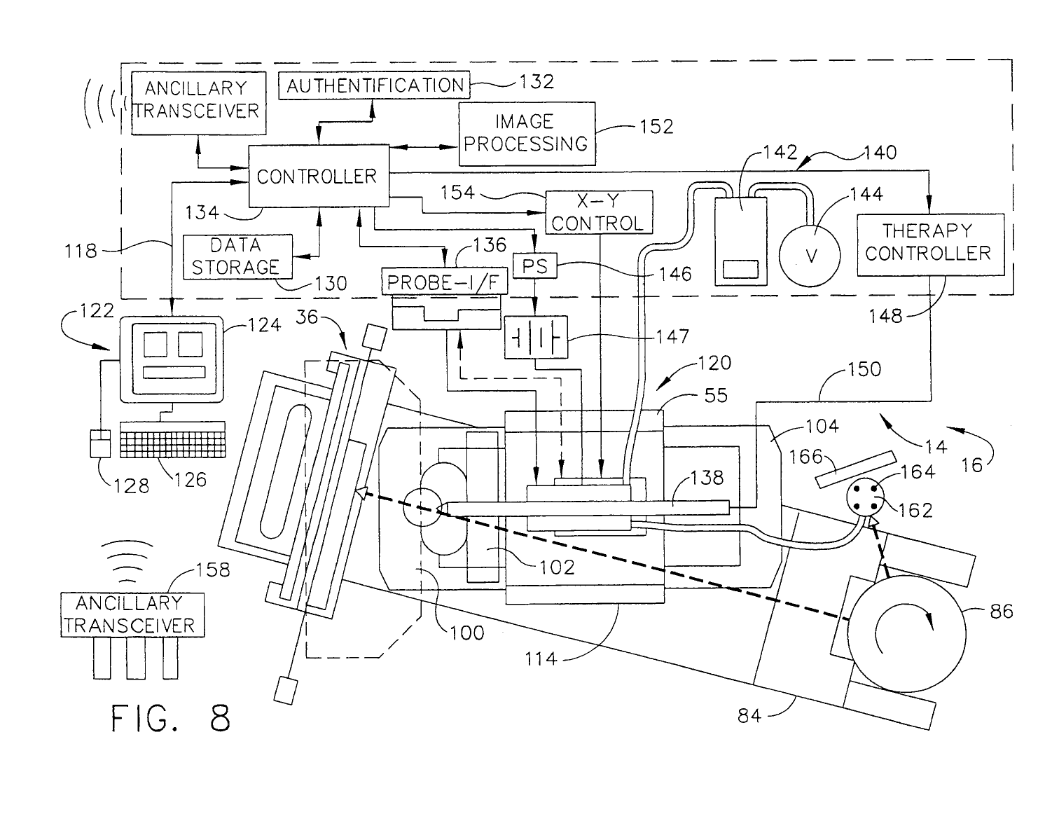

In FIG. 8, a fixed compression plate 100 and a compression paddle 102 movable toward and away from plate 100 are mounted above the C-arm 84 on an independently pivoted compression arm 104. Compression paddle 102 may be considered a biopsy compression device, since it incorporates both a transparent portion permitting X-rays to pass through it toward the patient's breast and image receptor 98, and a central needle access aperture, in the present example. The compression arm 104 of the present example also incorporates the movable table 55 for receiving the biopsy probe assembly 18 for performing a core biopsy procedure or a therapeutic treatment through the piercing lumen 30 without releasing the breast from the compression plate 100, thereby assuring that the target lesion coordinates determined by the original stereotactic measurements will be maintained upon insertion of the needle to reach the same target lesion coordinates.

Returning to FIGS. 4, 5 and 7, a central concave torso depression 106 surrounds the central aperture 96. Depression 106 provides comfortable support for the prone patient's head, shoulders and torso, with her hips and legs extending either to the right or to the left over the slightly higher end portions of platform 76, which may also incorporate left and right footrests 108, 110 if desired. The slight elevation of the patient's hips by depression 106 may maintain the normal relaxed curve of the patient's vertebra, while presenting a maximum volume of breast tissue through aperture 96 for X-ray examination. In addition, the slight elevation of the ends of platform 76 outside of the central depression 106 may provide underside clearance encircling aperture 96 for the upper end of X-ray tubehead 86 under platform 76. This may permit the focal point source (FP) of X-radiation to be elevated to a level nearly in tangent coincidence with the lower rim of aperture 96, providing exposure of a maximum volume of the patient's pendulant breast tissue for examination. Of course, these configurations are merely exemplary, and other alternative configurations may be used. Furthermore, different results may be obtained using the configuration of the present example.

In the present example, the front edge of platform 76 beside aperture 96, opposite pedestal 78, is formed as a removable panel cutout 112, providing unimpeded access beneath platform 76 for the radiologist and technicians, and permitting the patient's arm to be lowered through the open space left by the removal of a panel (not shown), possibly bringing her shoulder comfortably down toward the level of aperture 96, and possibly minimizing any distortion or stretching of the breast pendulant through aperture 96.

It should be appreciated that different positions of tubehead 86 may be produced by angular movement of C-arm 84 along a circular arcuate path. In the outermost position of the tubehead 86, X-radiation projected toward axis 88 may approach a lesion from the lateral aspect of the right breast or the medial aspect of the left breast if the patient's head is positioned to the right on platform 76. The left footrest 108 at the left end of platform 76 supports the patient's legs in this position, while the right footrest 110 at the right end of platform 76 may be retracted toward the table end. With the patient's head placed to the left of axis 88 and the right footrest 110 being extended from the right end of platform 76, X-radiation from tubehead 86 in its outermost position may approach the lateral aspect of the left breast or the medial aspect of the right breast. At either axial position, near the right end of platform 76 or near the left end of platform 76, the X-radiation may approach the breast from either above or below, with the image receptor 98 being positioned on the opposite side of the breast and the compression plate 100 and paddle 102, assuring that the patient is comfortably positioned with little risk of unexpected movement during the procedure.

In some examples, the tubehead 86 delivering X-rays to the patient will be positioned at the patient's head end of platform 76, with image receptor 98 and compression plate 100 being positioned on the underside of the pendulant breast and the compression paddle 102 being positioned on the upper side of the breast, both mounted on compression arm 104, which also provides support for the movable table 55 from this upper side when required. However, the presence of a lesion near the underside of the breast may indicate that the reverse orientation is desirable for minimum trauma, with the movable table 55 and compression paddle 102 being positioned on the underside of the breast with the X-ray tubehead 86 being positioned beyond compression plate 100 on the upper side of the breast. In this position, the entry of the biopsy needle 38 supported by the movable table 55 attached to compression paddle 102 into the underside of the breast tissue may offer the minimum path length for access to the lesion, and this position may be preferred by some patients to assure that any needle scar will be on the underside of the breast where it is less easily observed. Other suitable positions of components relative to each other and relative to a patient will be apparent to those of ordinary skill in the art.

Two additional tubehead positions being respectively displaced angularly by approximately 15 degrees counterclockwise and 15 degrees clockwise may be suitable angular displacements for stereotactic mammography. However, lesser angular amounts, of 10 degrees for example, or even greater angular amounts, on each side of the longitudinal axis of platform 76 can be used if desired, to assure that the stereoscopically displaced images both fall on the desired portion of the image receptor 98 of the electronic imaging optical system 90. Stereoscopic displacement of the lesion image may place it near the periphery of the total image plane in particular lesion orientations.

As shown in FIG. 8, X-, Y- and Z-axis indexing of the movable table 55 relative to the patient's breast tissue is provided in the present example by linear motorized adjustments of a supporting indexing carriage 114 movably mounted on linear bearings on the compression arm 104, pivoted on ledge 82 above pivoting C-arm 84. The indexing carriage 114 is manually or automatically positioned with the cooperation of a timing belt or endless chain drive, etc., to position the compression paddle 102 into gentle compressive contact with the patient's breast, clamping it gently but firmly against the fixed breast compression plate 100. The movable table 55 is also manually and/or automatically positioned relative to X, Y or Z coordinates to permit the operator to position the biopsy probe 38 as required by the lesion coordinates found by stereotactic X-ray observations or by other means.

In FIGS. 4-7, physical integration of a biopsy system 20 into a table 12 by magnetics, clips, etc. to hold accessories, control module 64, etc., may reduce hazards of inadvertent movement of these components during transit (to the extent that a vehicle 10 is used), may reduce tripping hazards, and may simplify interconnections to the diagnostic table 16 to the extent that function integration is desired. Of course, obtaining these results is not necessary, and some embodiments may fail to obtain any or all of these results. Similarly, other results may be obtained by some embodiments.

It will be appreciated that an integrated imaging, biopsy, and treatment system 14 may have a variety of additional components and/or properties. Several of such components and/or properties will be described in greater detail below, while other suitable components and/or properties will be apparent to those of ordinary skill in the art in view of the disclosure herein.

Integrated Device Control Interface

First, in some embodiments, an integrated device interface 118 integrates and controls a diagnostic station 16, including a biopsy system 120 (e.g., the biopsy system of FIG. 3, etc.) that may be assembled with or integral to a table 12 and a integrated imaging, biopsy and treatment system 14, through a single user interface 122. In other words, a single user interface 122 in communication with an integrated device interface 118 may be used to control operation of both a table 12 and a biopsy probe assembly 18, among other things. By way of example only, the functions that may be provided through a user interface 122 may include, but need not be limited to, the following: adjusting settings for biopsy probe assembly 18 (e.g., sampling speed, vacuum levels, etc.); performing diagnostics of integrated imaging, biopsy and treatment system 14 (e.g., diagnostics of individual components and/or of the system 14 as a whole, etc.); performing a test of the biopsy probe assembly 18 operability; arming or firing the piercer 20 and/or cutter tube 52; obtaining a tissue sample using biopsy probe assembly 18; performing a “clear probe” operation (e.g., clearing any tissue or debris from the piercer tube 54 and/or cutter tube 52, etc.), such as by using a vacuum, a saline flush, or some other means; inducing a vacuum within piercer 20 and/or cutter tube 52; opening/closing port 28; deploying a tissue marker through port 28; raising, lowering, rotating, or otherwise moving table 12; moving C-arm 84; operating optical system 90; positioning or moving compression plate 100, compression paddle 102, and/or compression arm 104; moving or positioning movable table 55; moving or positioning X-Ray tubehead 86; causing X-Ray tubehead 86 to emit X-rays; moving or otherwise adjusting indexing carriage 114; and/or performing any of the above-listed functions on any of the various other ancillary devices described elsewhere herein, among others. Still other components that may be controlled via a single user interface 122, and how such components may be controlled via user interface 122, will be apparent to those of ordinary skill in the art, including but not limited to any and all other components described herein, variations thereof, and suitable substitutes or supplements for such components.

Operator input (e.g., setup, control, actuation, positioning, etc.) for a biopsy system 120 may thus be accepted through the single user interface 122 and used to control the biopsy system 120. These control signals may include mechanical control signals (e.g., actuate firing or sampling of a needle), or electrical communication to system 120 to actuate similar functions or other functions. Thus, unlike a significantly autonomous control module 64 present in some biopsy systems 120, the mechanical motion necessary for positioning the piercer tube 24 and translating/rotating the cutter tube 52 may be provided by the diagnostic station 16 via the integrated user interface 122, reducing the mounting of the biopsy system 120 to only a disposable probe 138 portion. In other words, other components of a biopsy system 120 may remain permanently and integrally mounted relative to a table 12, with a disposable probe 130 removably mounted thereto.

In the present example, the single user interface 122 is depicted as a computer workstation with a monitor 124, keyboard 126, and graphical pointing device (e.g., mouse, etc.) 128. However, it will be appreciated that a single user interface 122 may have a variety of alternative components or configurations. For instance, user interface 122 may comprise commercial off the shelf (COTS) computer components coupled with an integrated device interface 118; a dedicated, customized, or proprietary user interface system (not shown) coupled with an integrated device interface 118; or any other suitable components, provided in any other suitable configurations. For instance, components forming a user interface 122 may be integrally mounted within a housing custom built for table 12.

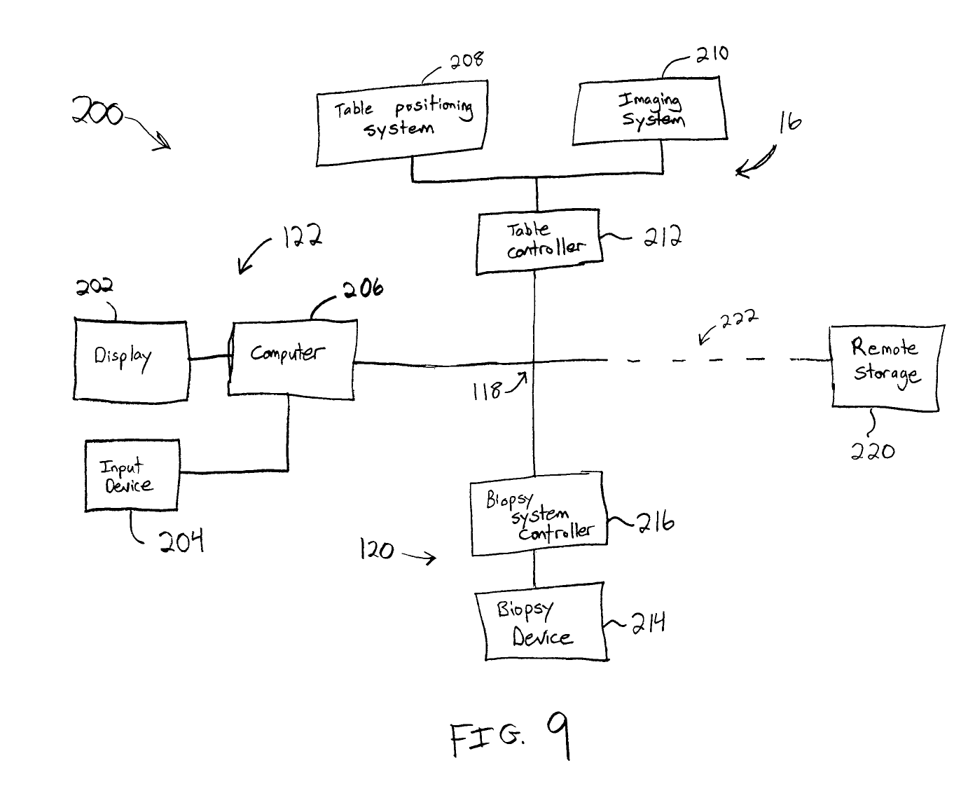

A block diagram of an exemplary integrated control system 200 is shown in FIG. 9. As shown, a user interface display 202 (e.g., a video monitor 124) and an input device 204 (e.g., keyboard 126, mouse 128, combinations thereof, etc.) are coupled with a control system computer 206. In this example, user interface display 202, input device 204 and control system computer 206 may collectively constitute a single user interface 122. As is also shown, control system computer 206 in this example is communicatively coupled with an integrated device interface 118. In addition, a table positioning system 208 and an imaging system 210 are coupled with a table controller 212, which is also communicatively coupled with the integrated device interface 118. Similarly, a biopsy device 214 is coupled with a biopsy system controller 216 to form a biopsy system 120, which is also communicatively coupled with the integrated device interface 118. User interface 122 is thus operable to control, via integrated device interface 118, table controller 212 to control table positioning system 208 and imaging system 201, as well as biopsy system controller 216 to control biopsy device 214. Other suitable components and arrangements that may be incorporated into an integrated control system 200 will be apparent to those of ordinary skill in the art. By way of example only, and as will be described in greater detail below, a remote storage 220 (e.g., server, etc.) may be communicatively coupled with integrated control system 200, such as via a network 222 (e.g., the internet, a dedicated network, a LAN or WAN, etc.).

In the present example, user interface 122 provides a broad range of control functions in addition to table 12 positioning controls, including operational displays and controls for a biopsy probe assembly 18, as described in U.S. Pat. No. 6,752,768, the disclosure of which is hereby incorporated by reference in its entirety. Of course, to the extent that other components or devices are included (e.g., devices other than a biopsy system 120 and/or table 12), control of such devices may also be integrated into a single user interface 122. Furthermore, control may be provided to such devices via one or more wires, wirelessly, or using combinations thereof. For instance, integrated device interface 118 may comprise one or more cables and/or a wireless communication hub, communicatively coupled with user interface 122.

Just as controls of various devices may be integrated through a single user interface 122, so may data obtainment, processing, and/or transfer, etc. Examples of data integration may include generating imaging, diagnostic, and/or treatment data stored in a patient record in a local data storage 130. Thereby, a large number of patients may be seen in a mobile setting when transmission of patient data is not available or under other circumstances.

In addition, a single user interface 122 may serve as a data gateway to local or remote institutional data repositories (e.g., one or more servers, etc.), such as a hospital laboratory information system (LIS) (not shown) or other remote storage 220, either in real-time, periodically, intermittently, or otherwise, regarding information such as the biopsy type, number of biopsy specimens, volume of tissue, patient ID, system ID, error logs, table data and/or operating parameters, etc. Such communication may be provided wirelessly (e.g., via satellite uplink, Wi-Fi, or some other modality or protocol of wireless communication) or otherwise. Data obtained through the system 200 may be reviewed by a physician, by some other person, and/or by a computer, at a remote location or elsewhere, in substantially real time, may be processed locally or remotely, and/or may simply be logged for archival purposes. Similarly, to the extent that the system 200 is immobily provided in a substantially fixed location or facility (e.g., within a hospital, not in a mobile truck 10, etc.), the integrated control system 200 may be communicatively coupled with a LAN or WAN within the facility. For instance, the system 200 may be communicatively coupled with a hospital's internal network or information system. Such communication may be provided via wire (e.g., Ethernet cable, etc.), wirelessly, or combinations thereof. Other ways in which data or commands may be communicated and processed will be apparent to those of ordinary skill in the art.

Furthermore, just as data may be transferred from the system 200 to another location (e.g., to a local or remote location), data and/or commands may also be transferred from such a second location to the system 200 of the present example. For instance, a table 12 and/or biopsy system 120 may be controlled at least in part by a remote operator. Such remote operation may be performed by a human and/or automatically. Furthermore, operation may be performed by a person co-located with the system 200 as well as by a person located remotely relative to the system 200 of the present example.

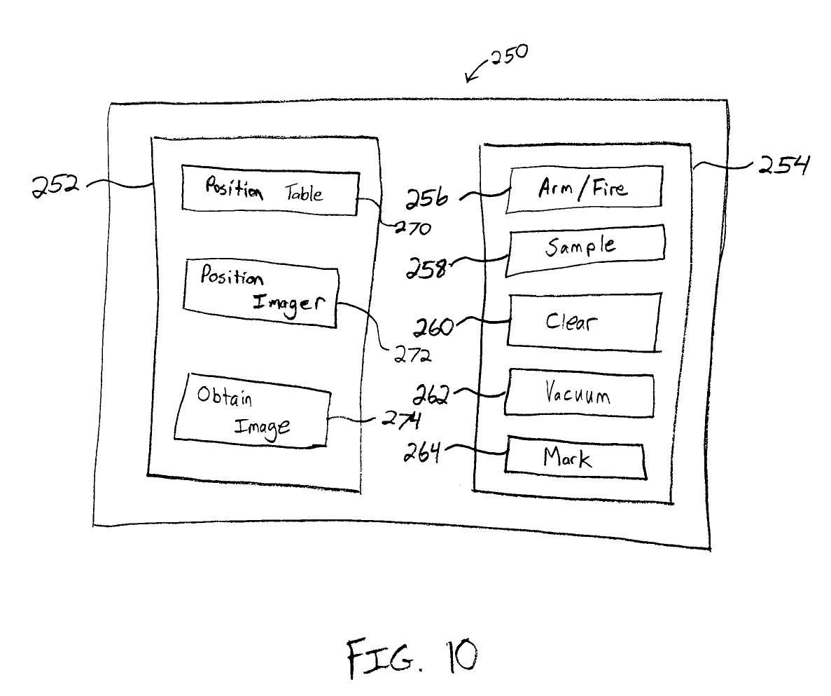

It should be appreciated with the benefit of the present disclosure that data associated with the type, identification, operational information, status, and so on of the biopsy system 120 may be displayed upon the single user interface 122. A merely exemplary graphical user interface (GUI) 250 that may be provided on user interface 122 is shown in FIG. 10. As shown, the GUI 250 provides a table control and imaging function frame 252 and a biopsy control frame 254. The table control and imaging function frame 252 of this example includes: a feature 270 to reposition the table 12, a feature 272 to reposition an imaging system 210, and a feature 274 to obtain an image using the imaging system 210. The biopsy control frame 254 of this example includes: a feature 256 to arm/fire a probe 138 or other part of a biopsy device 214, a feature 258 to obtain a tissue sample with biopsy device 214, a feature 260 to clear the biopsy device 214 (e.g., to evacuate a tissue sample or debris from the biopsy device 214), a feature 262 to induce a vacuum within the biopsy device 214, and a feature 264 to deliver a marker through the biopsy device 214. Of course, these frames and features are merely exemplary, and any other suitable frames and/or features may be provided through a GUI 250 in any other suitable arrangement.

Furthermore, a single user interface 122 may present multiple GUI's. For instance, one GUI 250 may permit a user to select which component(s) they would like to control, such that a user's selection will then call up a second GUI 250 that is dedicated to the component(s) that the user has indicated they would like to control. In some embodiments, when a user indicates that they want to control a biopsy device 214, the user interface 122 may call up a GUI 250 similar to any of those shown or described in described in U.S. Pat. No. 6,752,768, the disclosure of which has been incorporated by reference herein. Of course, any other suitable GUI 250 or GUI's 250 may be used to control a biopsy device 214. As another merely illustrative example, activation of feature 270 to reposition the table 12 may call up another window, screen, or frame (not shown) permitting specific commands for table 12 positioning, and/or table 12 position information. It will also be appreciated that a user may interact with a GUI 250 using a mouse 128, using touch-screen technology, and/or using any other suitable device, technique, or technology, including combinations thereof.

In view of the foregoing, those of ordinary skill in the art will appreciate that the control of and data obtained using a diagnostic station 16 and biopsy device 214 may be integrated into a single user control system 200 having a single user interface 122. However, the above described components, configurations, arrangements, and functionalities of a control system 200 are merely exemplary, and the inventors contemplate that a control system 200 may be carried out in a variety of alternative ways, including but not limited to various other components, arrangements, and methods of operation.

Ancillary Device Identification and Authentication

Second, in some embodiments, an ancillary device authentication system 132 may include a device or programming object in communication with or integral to the single user interface 122 and/or a controller 134 of the diagnostic station 16. In some embodiments, components of the integrated imaging, biopsy and treatment system 14 may be assembled and disassembled due to use of disposable components for sterility or other purposes. The diagnostic station 16 may communicate (e.g., bi-directionally, one-way, etc.) with an ancillary device (e.g., the biopsy system 120, biopsy device 214, biopsy probe 18, etc.) to either prevent the use of unauthorized devices, or to limit features available to unauthorized devices. For example, a specific, proprietary communication protocol or handshake may be employed to ensure that only those devices approved or certified by the table manufacturer are physically connected, are allowed to work in conjunction with the diagnostic station 16, or are allowed access to specific integrated features (e.g., software, display features, etc.) of the diagnostic station 16. A proprietary communication protocol or handshake may be implemented via one or more wires, wirelessly, or otherwise. Furthermore, proprietary physical connectors may be used for electronic communication, as described in greater detail below.

It should be appreciated with the benefit of the present disclosure that this authorization may indicate the model or manufacturer of the ancillary device, or verify that the device complies with relevant standards or diagnostic table manufacturer requirements, or provide or be based on other information in any suitable fashion. For instance, the authorization may be implemented as an automatic transmission of data upon connection, or the response of a biopsy device 214 to an electronic query or handshake from the diagnostic table controller 212. It could also take the form of a proprietary handshake or encrypted data.

In the present example, if the ancillary device is properly authorized by the diagnostic station 16, the diagnostic station 16 allows the ancillary device to function with the table 12. If the ancillary device cannot be authorized, the table 12 will not allow the ancillary device to fully function with the table 12. This could include providing “tiered” access, where certain devices can access a full set of functions within a first tier, a second tier providing a smaller set of functions, and further tiers providing even more reduced sets of functions, perhaps including to a tier providing zero functionality for the ancillary device (and/or zero functionality for other components of the system 200 when an unauthorized ancillary device is coupled with the system 200, etc.). This scheme may also be limited to granting access to certain table features (e.g., display modes or other integrated operational modes, etc.), while allowing even non-authorized devices some basic functionality. This scheme could also prevent the use of the diagnostic station 16 at all when a non-authorized device is anchored. Table “functionality” may include the physical ability to mount to the table 12, and the ability of the diagnostic station 16 or at least one component thereof to perform some or all of its normally intended functions, among other functions.

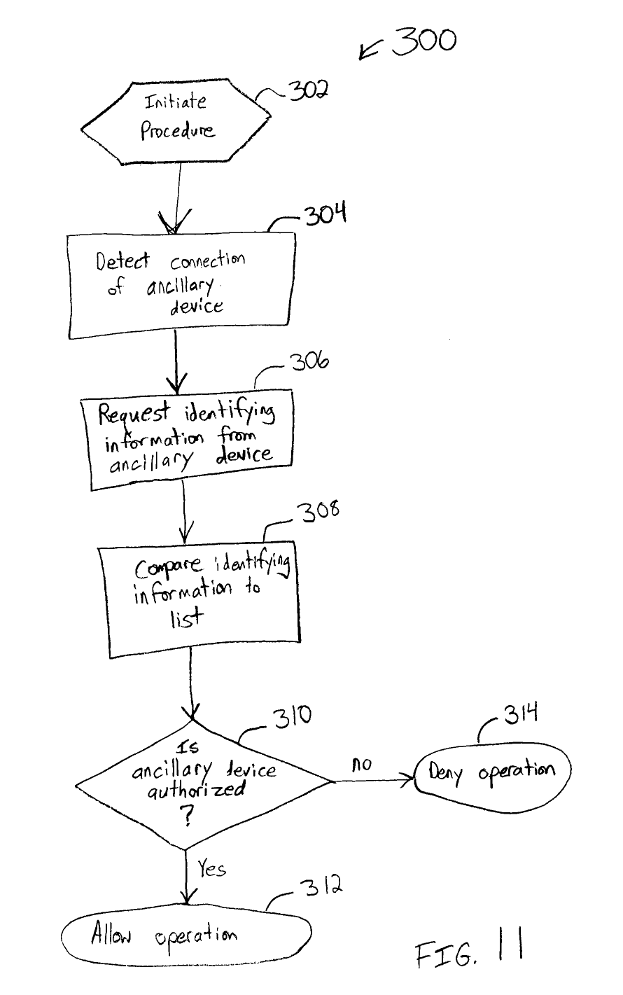

One merely exemplary identification and authentication routine 300 that may be performed by a system 200 is illustrated in FIG. 11. In this example, as shown in block 302, the procedure is initiated. By way of example only, this may be accomplished simply by turning on one or more components of the system 200, such that the system 200 is on “standby” to perform subsequent steps of the routine 300. As shown in block 304, the connection of an ancillary device (e.g., a biopsy device 214) is detected. As will be apparent to those of ordinary skill in the art, such connection may be detected in any number of a variety of ways. For instance, a user may manually activate a switch (not shown) to indicate to the system 200 that an ancillary device has been connected. Alternatively, a component of the system 200 and/or the ancillary device itself may have a feature (e.g., a sensor, switch-engaging feature, etc.) that is configured to automatically detect and/or communicate the connection of the ancillary device to the system 200. Other ways in which connection of an ancillary device to a system 200 may be detected will be apparent to those of ordinary skill in the art.

Next, as shown in block 306, identifying information is requested from the ancillary device. By way of example only, such information may be requested from and/or obtained from a biopsy system 120, a biopsy system controller 216, and/or a biopsy device 214. After such information is obtained, the identifying information is compared to a list, as shown in block 308. This comparison yields whether the ancillary device is authorized, as shown in block 310. As will be apparent to those of ordinary skill in the art, there are a variety of ways in which identifying information may be compared to a list, as shown in blocks 308 and 310. By way of example only, a list of identifying information associated with authorized ancillary devices (and/or unauthorized ancillary devices) may be stored locally (e.g., within control system computer 206, etc.), anywhere in communication with the network 222 (e.g., a computer or server within a hospital that the system 200 resides in, etc.), within an external database (e.g., within remote storage 220, etc.), within a removable device coupled with the system 200 (e.g., in a CD or flash/USB drive coupled with a control system computer 206, etc.), or elsewhere, including combinations of such locations. In another variation, an internet-based compatibility list is provided and interrogated by the system 200. Similarly, just as a list or other source of information may be stored and accessed locally or remotely, etc., an act of comparing the identifying information 308 may be performed locally and/or remotely, etc. For instance, the comparison 308 may be performed on or within the same device in which the list or other source of information is stored, or in any other suitable location, including combinations thereof.

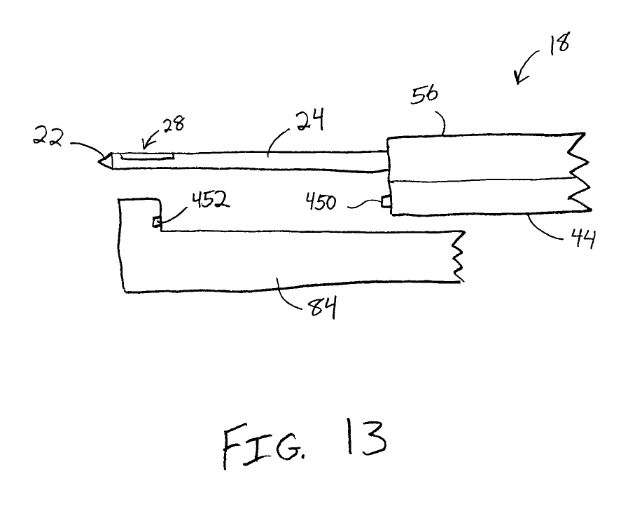

It will also be appreciated that a number of modalities may be used to obtain identifying information from an ancillary device. For instance, en electronic query/response may be used (e.g., using a standard serial connection, Ethernet, Bluetooth, etc.) between system 200 and the ancillary device to obtain a serial number, passcode, manufacturer name or code, model number, features, etc. Alternatively, a mechanical connection may be used, including but not limited to the type described below with reference to FIG. 13. Other ways in which identifying information may be obtained and/or processed to determine authentication will be apparent to those of ordinary skill in the art.

If the ancillary device is authorized (e.g. pursuant to the step shown in block 310), full operability of the ancillary device may be permitted, as shown in block 312. If the ancillary device is not authorized, full operability of the device may be denied, as shown in block 314. By way of example only, full operability of the ancillary device may be denied, as shown in block 314, under any of the following conditions or combinations of such conditions: the system 200 was unable to obtain any identifying information from or regarding the ancillary device in block 306; the identifying information did not match with any information on the list in the comparison of block 308; the identifying information matched with information associated with an unauthorized device in the comparison of block 308; the system 200 has determined that an ancillary device that should only be used once has already been used at least once; or under any other circumstances. Furthermore, as an alternative to denying operation of an unauthorized ancillary device altogether, the system 200 may permit certain functions while denying others, as described elsewhere herein.

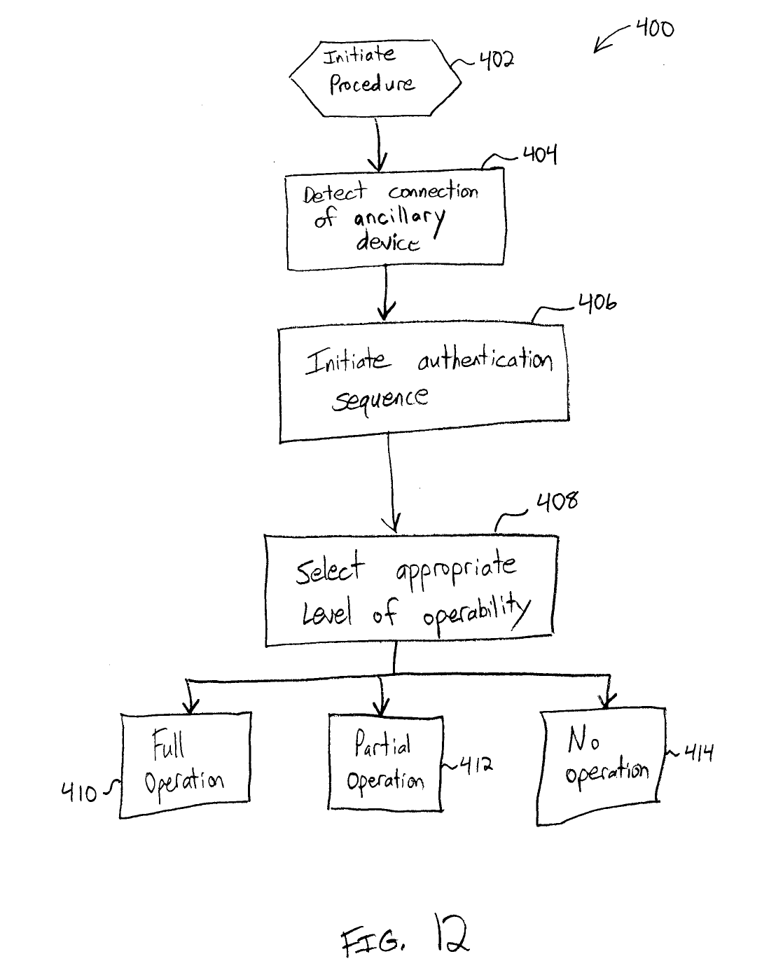

Another merely exemplary authentication routine 400 that may be performed by a system 200 is illustrated in FIG. 12. In this example, as shown in block 402, the procedure is initiated. As noted above, and by way of example only, this may be accomplished simply by turning on one or more components of the system 200, such that the system 200 is on “standby” to perform subsequent steps of the routine 400. As shown in block 404, the connection of an ancillary device (e.g., a biopsy device 214) is detected. As will be apparent to those of ordinary skill in the art, and as noted above, such connection may be detected in any number of a variety of ways. For instance, a user may manually activate a switch (not shown) to indicate to the system 200 that an ancillary device has been connected. Alternatively, a component of the system 200 and/or the ancillary device itself may have a feature (e.g., a sensor, a switch-engaging feature, etc.) that is configured to automatically detect and/or communicate the connection of the ancillary device to the system 200. Other ways in which connection of an ancillary device to a system 200 may be detected will be apparent to those of ordinary skill in the art.

Next, as shown in block 406, an authentication sequence is initiated. As with other merely exemplary steps described herein, this step 406 may be carried out in any number of a variety of ways. For instance, an authentication sequence may be initiated through an identification request and comparison procedure as described above with respect to blocks 306, 308, and 310 of routine 300. Alternatively, an authentication sequence may include entry of a password, which can be unique or standard (e.g., entered by a user via user interface 122, entered by a user via the ancillary device, entered automatically by the ancillary device, etc.); a query/response between the system 200 and the ancillary device (e.g., system 200 seeks a particular type/content or form/format of response from an ancillary device, etc.); a particular encryption of communication or encrypted data interchange to and/or from the system 200 and/or the ancillary device; a specific handshake between the system 200 and the ancillary device; a proprietary or otherwise specific communication protocol between the system 200 and the ancillary device (e.g., commands and/or responses are fully customized for system 200, etc.); a physical feature or interlock of the system 200 and the ancillary device (e.g., complimentary physical features or structures of a component of the system 200 and the ancillary device, etc.); and/or an “authentication key” (e.g., a certain key, data, or device that must be present in a biopsy system 120, such as a VPN token used for network access or a specific RFID tag, etc.). Still other ways in which an authentication sequence may be initiated and/or carried out in accordance with block 406 will be apparent to those of ordinary skill in the art.

Proceeding further in the routine 400 of the present example, based on the authentication sequence, an appropriate level of operability is selected, as shown in block 408. As shown, the selected level of operability may include full operation 410, partial operation 412, or no operation 414. In this context, the selected level of operability may relate to operability of the ancillary device only and/or operability of other components of the system 200. For instance, some or all of the components of the system 200 may be fully operable before an ancillary device is connected, yet some or all of such components (e.g., a table 12, etc.) may be rendered only partially operable (block 412) or completely inoperable (block 414) when an ancillary device has been connected that is partially authenticated or that is not authenticated (e.g., unauthorized, etc.). Similarly, some or all of the components of the system 200 may be completely or partially inoperable unless and until a fully or partially authorized ancillary device is connected with the system 200. By way of example only, the user interface 122 of the system 200 may be rendered inoperable while other components of the system 200 may remain operable when an unauthorized or partially authorized ancillary device is connected with the system 200. Other ways in which one or more (e.g., all) components of a system 200 may be rendered fully or partially inoperable will be apparent to those of ordinary skill in the art.

Just as operability of the system 200 or some of its components may be affected by the authentication sequence (block 406) and the selected level of operability (block 408), so may the operability of the ancillary device (e.g., in addition to or as an alternative to the operability of the system 200 or one or more of its components being affected). For instance, a biopsy device 214 or one or more components thereof may be rendered inoperable as coupled with a system 200 when it is determined that the biopsy device 214 is not authentic (e.g., unauthorized, etc.) or is only partially authenticated or authorized. Suitable ways in which an ancillary device may be rendered wholly or partially inoperable, including but not limited to combinations of such inoperability with any full or partial inoperability effected upon the system 200 or one or more components thereof, will be apparent to those of ordinary skill in the art.

Alternatively or in addition to the authentication discussed above, an authenticating connection may be physical rather than electronic. In some embodiments, a physical adapter or interface (not shown) between the table 12 and biopsy device 214 may has a proprietary shape or structural feature or configuration that allows only a certain device (e.g., one having a complimentary shape or structural feature) to be connected. By way of example only, a physical adapter or interface between the table 12 and biopsy device 214 may include complimentary shapes or profiles that are dovetailed, “T”-shaped, etc. Alternatively or in addition, a physical adapter or interface between the table 12 and biopsy device 214 may provide an input (e.g., magnetic sensor, switch closing, etc.) when a device with the necessary form factor is attached. One merely illustrative example of such a feature is shown in FIG. 13. In this particular example, the ancillary device is a biopsy probe 18 that includes a base 44 for coupling with a portion of a C-arm 84 in a diagnostic station 16. As shown, the base 44 has a male feature 450 that is configured to activate a microswitch. Alternatively, male feature 450 may be provided on the upper portion 56 of biopsy probe 18 or elsewhere. C-arm 84 of the present example has a complimentary female feature 452 that includes a microswitch that is configured to be engaged by male feature 450. In particular, when biopsy probe 18 is fully engaged with C-arm 84, male feature 450 engages female feature 452 to close the microswitch. Closure of the microswitch may be required in order for system 200 and/or biopsy probe 18 to be fully operable. In addition or in the alternative, an adaptor or connector could also include a proprietary electronic connector. Other ways in which an authenticating physical connection may be provided will be apparent to those of ordinary skill in the art.

Similarly, in some embodiments, an automatic ancillary device identifier 136 facilitates automatic identification and/or calibration of an ancillary device mounted to the diagnostic station 16, such as a biopsy probe 138 of the biopsy system 120 of a certain length. Using an embedded memory or processor, the station 16 interrogates and identifies the device 138. The diagnostic station 16 may make decisions about whether or not the device 138 can be used. For instance, serial number identification as well as product type and/or use history may be referenced for purposes of avoiding possibly dangerous reuse of a previously used disposable component. Unrecognized product types may be prevented from use, or only have limited uses available, to avoid incompatible or unvalidated combinations.

It will be appreciated that disposable articles that are appropriate for the installed equipment may be stored integral to the table 12 with an inventory maintained. In order to prevent use of an incorrect type of disposable component, the proper inventory item may be identified based upon a procedure that is user selected and/or by identification of an installed component. This proper disposable component may further be automatically dispensed by the diagnostic station 16. For repeated operations, a disposable unit (not shown) for biohazards may further be mounted or integrated with the diagnostic station 16, prompting the user to throw away such components after use, prior to moving on to other activities, to minimize contact hazards. Furthermore, in some embodiments, each individual ancillary device carries a unique identification, such that use of each individual ancillary device may be logged and tracked. In such embodiments, the system 200 may track use of a given individual ancillary device. To the extent that such an ancillary device is only supposed to be used once (e.g., disposable device, etc.), the system 200 may prevent subsequent use of such a device. Similarly, to the extent that a disposable unit for biohazards is be mounted or integrated with the diagnostic station 16, use of such a disposable unit may be monitored. For instance, where the system 200 detects that a disposable ancillary device or component has been used, the system 200 may prevent operability of at least a portion of the system 200 until it detects that the disposable ancillary device or component has been properly disposed of in the disposable unit. Suitable techniques for carrying such procedures out will be apparent to those of ordinary skill in the art, as will other ways in which a system 200 may handle single-use (e.g., disposable) devices or components.

Referring back to FIG. 9, in some embodiments, ancillary device identification and/or authentication (e.g., routine 300 and/or routine 400) may be performed, at least in part, within user interface 122. In addition or in the alternative, ancillary device identification and/or authentication may be performed, at least in part, in a remote device 220 (e.g., via network 222, etc.). For instance, a list of identifying information for authentic or permissible devices may be stored in a remote device 220 and referenced therefrom. Other suitable locations where ancillary device identification and/or authentication may be performed, including combinations of such locations, will be apparent to those of ordinary skill in the art. Furthermore, it will be appreciated that results of ancillary device identification and/or authentication may be rendered, at least in part, on a user interface display 202. For instance, a user interface display 202 may display the model type/number and manufacturer of an ancillary device that is coupled with the system 200. Furthermore, a user interface display 202 may display a message indicating whether the ancillary device is authentic, whether full functions will be permitted (e.g., based on authentication, etc.), or other identification/authentication related information. A user interface display 202 may also render information regarding the use of an ancillary device (e.g., how many times it has been used, when it was used, etc.), and/or even information broken down based on particular components of such a device. Other types of information that may be displayed on a user interface display 202 before, during, and/or after ancillary device identification and/or authentication will be apparent to those of ordinary skill in the art.

Integrated Utilities

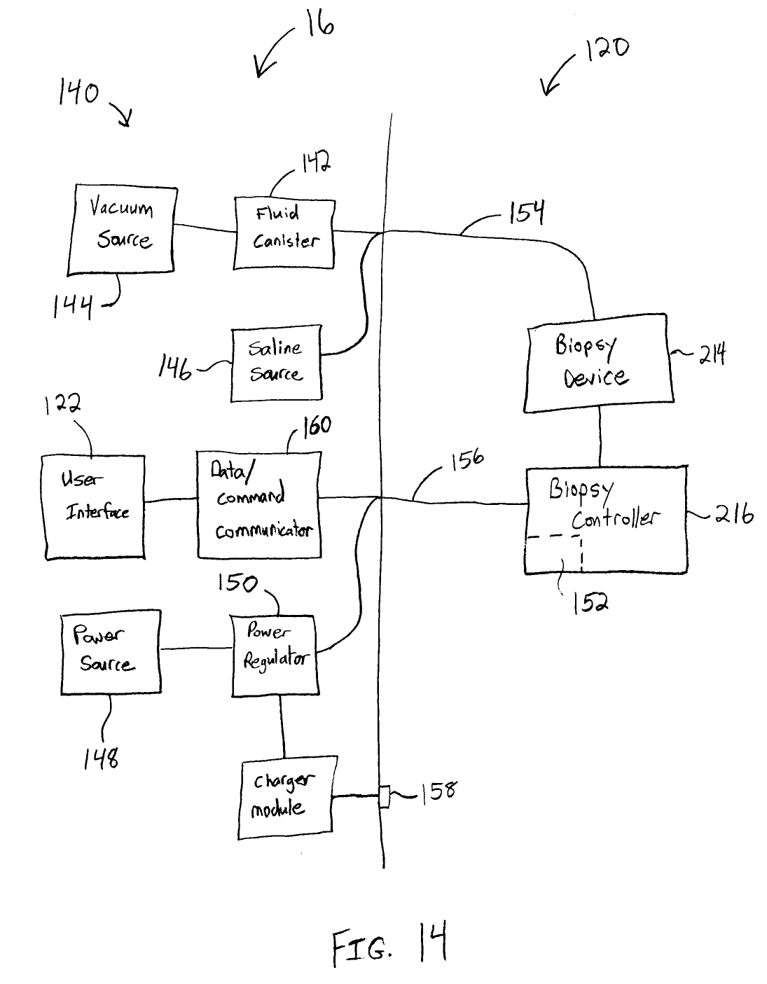

Third, in some embodiments, a merely illustrative example of which is depicted in FIG. 14, a diagnostic station 16 may include integrated utilities 140, including fluid capture vessel 142 (e.g., a canister, bag or pouch, etc.), fluid and vacuum supply 144 (e.g., vacuum canisters, vacuum pump, pass-through or regulated conduits that may be attached to a wall vacuum port, etc.), thereby minimizing space requirements and reducing the likelihood of inadvertent exposure of personnel to potentially contaminated biohazards. The fluid capture and vacuum capabilities may be advantageously readily detachable for repair, replacement, and cleaning.

Integrated utilities 140 may also include a saline supply 146 (or supply of any other type of fluid) for flushing of a biopsy probe 138 or for other purposes. Of course, in some embodiments, saline supply 146, and/or vacuum supply 144, among other components, may be omitted. In the present example, however, vacuum supply 144, fluid capture vessel 142 and saline supply 146 are in communication with biopsy device 214 via a multi-lumen conduit 154. Wile multi-lumen conduit 154 of the present example has a unitary construction, alternative embodiments may use a plurality of separate and discrete conduits to provide fluid communication. A multi-lumen conduit 154 and/or connectors for a multi-lumen conduit 154 may be proprietary (e.g., to prevent unauthorized couplings) or may be formed of commercial off the shelf conduit, etc. Various ways in which a multi-lumen conduit 154 may be configured, and ways in which a multi-lumen conduit 154 may be coupled with diagnostic station 16 and biopsy device 214, will be apparent to those of ordinary skill in the art.

Integrated utilities 140 may further include an electrical power supply (PS) 148 with the desired voltage regulation through a regulator 150. As shown, power supply 148 is provided within diagnostic station 16, but in other embodiments, power is provided by an external source or adapter (e.g., through an adapter mounted to table 12, etc.). At least one cable 156 may be used to connect a biopsy controller 216 with power regulator 150 in order to provide power to biopsy device 214. For instance, biopsy device 214 may draw power from power source 148 via cable 156 during use of biopsy device 214. Alternatively or in addition, as described in greater detail below, biopsy controller 216 may draw power from power source 148 via cable 156 when biopsy device 214 is not in use, such as to charge a battery supply 152.

In some embodiments, a more autonomous, legacy ancillary device, depicted as the biopsy system 120, may include a battery power supply 152 that would be trickle charged by the power supply 148 of the diagnostic station 16 via cable 156. Although such a battery power supply 152 may be integral to the biopsy system 120, the battery power supply 152 may comprise a replacement battery module. One or more battery receptacles 158 may be formed into the diagnostic station 16 such that one or more replacement batteries 152 may be charged in advance and are readily locatable.

As is also shown in FIG. 14, a data/command communicator 160 may be integral with diagnostic station 16. For instance, data/command communicator 160 may serve as a relay or intermediary between user interface 122 and biopsy controller 216. As is also shown, data and/or commands that are provided through data/command communicator 150 may be communicated to and/or from biopsy controller 216 via cable 156. Of course, data and/or commands may be communicated wirelessly instead, such as by any of the wireless communication structures or techniques described herein, among others. It will also be appreciated that cable 156 and diagnostic station 16 may be provided with proprietary or custom connectors (not shown), such that standard off the shelf cables cannot be used for cable 156. For instance, the configuration and operability of cable 156 may be provided in a manner to ensure authentication of cable 156 that may be used to couple a biopsy system 120 or other ancillary device with diagnostic table 16.

Of course, the integrated utilities 140 explicitly noted above and illustrated in FIG. 14 are merely exemplary, and it will be appreciated that any of those utilities 140 may be omitted, substituted, or supplemented as desired. Furthermore, other types of integrated utilities 140 may be provided. By way of example only, one or more mechanical utilities (not shown), such as a source of mechanical power (e.g., direct drive) in lieu of or in addition to electrical power, may be provided as an integrated utility 140. Another merely exemplary integrated utility 140 may include a source of pressurized air (not shown), such as to power a pneumatic biopsy device or other device. Additional integrated utilities 140 may include a mounting location or pump (not shown) for liquids, such as therapeutic liquids, or a source of hydraulic power. Other suitable utilities 140 that may be integrated with a diagnostic station 16 will be apparent to those of ordinary skill in the art. Those of ordinary skill in the art will also appreciate that one or more integrated utilities 140 may be permanently mounted on or in diagnostic station 16, or may be removable and/or accessible for replenishment (e.g. saline, vacuum canisters, etc.) or maintenance.

It will be appreciated that operability of one or more of the above-noted integrated utilities 140 may be restricted, at least in part, based on ancillary device identification and/or authentication, such as the identification and authentication routines 300, 400 discussed above, or based on other considerations or processes.

Integrated Surgical, Therapy, and Diagnostic Devices

Fourth, in some embodiments, an integrated imaging, biopsy and treatment system 14 includes a therapy controller 148 and an active treatment element 150, which may create a surgical effect, a therapeutic effect, and/or a diagnostic effect. By way of example only, a surgical element that may be integrated into the system 200 may include one that is operable to coagulate, remove, or otherwise perform a surgical function on tissue, etc. For instance, examples of treatment systems may include devices using radio frequency (e.g., VALLEYLAB FORCE FX general purpose RF generators, etc.), laser (e.g., Lumenis Versapulse laser system, etc.), ultrasound, microwave (e.g., VIVAWAVE microwave system), ultrasonics (e.g., ETHICON ENDO-SURGERY, Cincinnati, Ohio Harmonic Scalpel generator), high intensity focused ultrasound (HIFU) (e.g., SONOBLATE system), etc., any or all of which may be integrated or incorporated into the diagnostic station 16 or otherwise integrated or incorporated into the system 200 in accordance with the teachings herein.

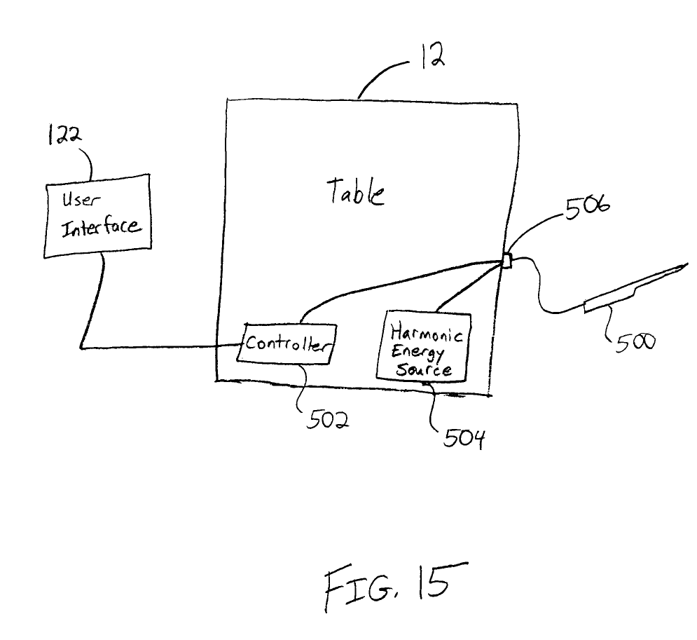

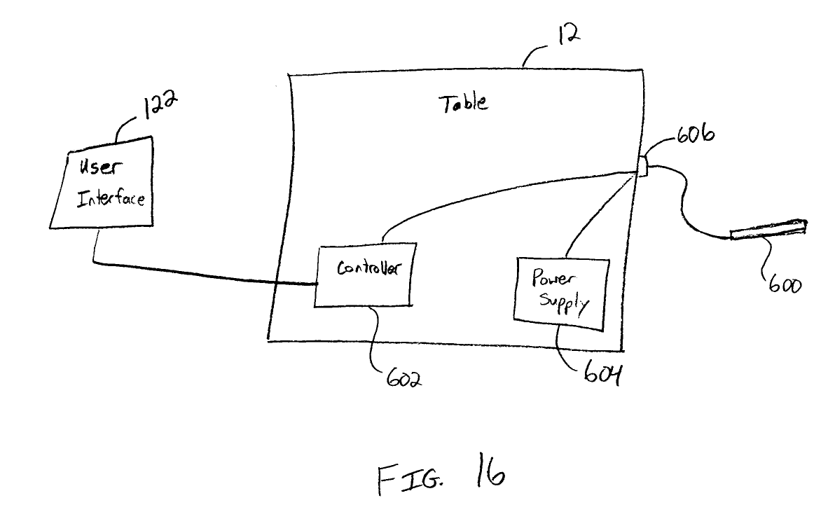

By way of illustration, an example of a harmonic device 500 (e.g., harmonic scalpel) being incorporated into the system 200 is illustrated in FIG. 15. As shown, a controller 502 and a harmonic energy source 504 are integral with a table 12. A harmonic device 500 is in communication with controller 502 and harmonic energy source 504 via a proprietary connector 506. User control of harmonic device 500 is provided via the integrated user interface 122, which is communicatively coupled with controller 502. Other ways in which a harmonic device 500 (or any other surgical device) may be integrally incorporated into a system 200 will be apparent to those of ordinary skill in the art. Furthermore, identification and/or authentication of a harmonic device 500 (or any other surgical device) may be provided in accordance with routines 300, 400 described herein or otherwise, thereby regulating operability of such devices based on identification and/or authentication.

Merely illustrative examples of therapy elements that may be integrated into the system 200 may include those that are operable to provide ablation (e.g., cryoablation, RF ablation, etc.), conductive thermal energy (e.g., THERMACHOICE by Johnson & Johnson), irradiation, such as traditional brachytherapy systems, and interstitial X-ray systems such as the AXXENT electronic brachytherapy system by Xoft Inc., etc. One such example is shown in FIG. 16. In this example, a controller 602 and a power supply 604 are integral with a table 12. An interstitial X-ray brachytherapy end effector 600 (e.g., a disposable surgical x-ray tube) is in communication with controller 602 and power supply 604 via a proprietary connector 606. User control of brachytherapy end effector 600 is provided via the integrated user interface 122, which is communicatively coupled with controller 602. Other ways in which a brachytherapy end effector 600 (or any other therapeutic device) may be integrally incorporated into a system 200 will be apparent to those of ordinary skill in the art. Furthermore, identification and/or authentication of a brachytherapy end effector 600 (or any other therapeutic device) may be provided in accordance with routines 300, 400 described herein or otherwise, thereby regulating operability of such devices based on identification and/or authentication.

In the present example, the controller 134 references an image processing unit 152 to analyze the images produced for generating spatial coordinates for directing an X-Y control 154 that positions the movable table 55 of the biopsy system 120. The diagnostic image is produced from an X-ray source, depicted as the tube head 86. An active treatment element 150 may be guided to these spatial coordinates accordingly, before, during, or after guidance of a biopsy device 214 to such coordinates, or even where a biopsy device 214 is not provided or otherwise used. For instance, as described in greater detail below, an active treatment element 150 may be configured to cooperate with a biopsy device 214, such that at least a portion of the active treatment element 150 may be introduced through a lumen of the biopsy device 214 that has been inserted into a patient. In other words, an active treatment element 150 may be positioned at a biopsy site without having to be separately targeted and/or guided to the biopsy site. Of course, while targeting in the present example is provided through diagnostic imaging produced from an X-ray source, it will be appreciated that targeting may be based on any other form of imaging or using any other suitable techniques and/or coordinate systems.

A biopsy system 120 may also provide a through-lumen or have a detachable back portion to leave the biopsy probe 138 in place for follow-on treatment through the access provided to the tissue in the biopsy site, complimenting the already synergistic capabilities of a mobile treatment facility or an otherwise integrated system 200. Illustrative examples of such biopsy probes 138 are described in U.S. Pub. Nos. 2003/0199754, entitled “Method for using an MRI Compatible Biopsy Device with Detachable Probe;” and 2005/0277829, entitled “MRI Biopsy Apparatus Incorporating a Sleeve and Multi-function Obturator,” the disclosures of which are hereby incorporated by reference in their entirety. By way of example only, such treatment may include insertion of an X-ray tube stylet, such as for performing electronic interstitial brachytherapy, sized for insertion through the biopsy probe 138; temporary disposal of a radioactive element in tissue for brachytherapy; disposal of one or more markers to mark the site of the biopsy; excision of tissue if pathology determines that the biopsy was cancerous; post-operative drainage of biopsy site; interstitial laser treatment; RF treatment; cryotherapy; etc. Other procedures that may be performed through a left-in biopsy probe 138 will be apparent to those of ordinary skill in the art.

While in some embodiments, a biopsy probe 138 is left in place (e.g., still inserted in a patient) while the rest of the biopsy device 214 is removed to permit proximal access through the biopsy probe 138, in other embodiments, the biopsy device 214 is configured to permit access to the biopsy site through the biopsy probe 138 without requiring any components of the biopsy device 214 to be detached from the biopsy probe 138. For instance, a lumen may extend from the side aperture 28 all the way to the proximal end or a proximal portion of the biopsy device 214 in order to permit access to the biopsy site from the proximal end or portion of the biopsy device 214. In such embodiments, a biopsy device 214 may include a movable cover or other feature to permit full proximal access to such a lumen. It will be appreciated that, in some instances, when a biopsy probe 138 is left in place within a patient after the rest of a biopsy device 214 has been removed, the biopsy probe 138 may no longer be fixed relative to the table 12 (e.g., the remainder of biopsy device 214 remains fixed to table 12, but not the probe 138), such that a patient may be repositioned to facilitate treatment or other processes through probe 138. Other ways in which access to a biopsy site may be permitted through a lumen (e.g., through a piercer lumen 30 extending through a piercer tube 24 that remains inserted in a patient before or after a biopsy sample has been taken) will be apparent to those of ordinary skill in the art.

As suggested above, in some embodiments, a therapeutic agent or device is introduced through a lumen of a biopsy device 214 that is left inserted in a patient (e.g., within a cavity left after a biopsy sample is taken). For example, in some instances, there may be benefits to providing a therapeutic dose of ionizing radiation or other therapeutic agent to specific tissue (e.g., to irradiate a tumor). An example of such as device is the AXXENT interstitial X-ray system from Xoft Inc., which provides an intersitital x-ray tube to irradiate targeted tissue. Precise placement of such treatment directly to the affected tissue without damaging healthy tissue may have particular advantages in some situations, such as if a second procedure is avoided following a biopsy procedure. Combining tissue biopsy and therapeutic treatment may provide a simpler, more integrated, and more effective system, reducing the need to re-target tissue after biopsy under certain circumstances. The therapeutic device or agent may be introduced through the lumen of the piercer tube 24, into the cavity left by the biopsy, allowing the therapy to be targeted to the suspicious tissue. For example, the AXXENT interstitial X-ray system from Xoft Inc. includes an interstitial source of therapeutic X-rays. This source could be deployed through the piercer tube 24 to access the targeted tissue. Without being so introduced, the tissue may need to otherwise be re-targeted during a later therapeutic procedure, potentially adding cost and potentially unnecessary X-ray exposure to the patient.

Alternatively, a device for administering a local anesthetic or other fluid or material may be introduced in such a lumen, before, during, or after a biopsy being performed.

Similarly, some embodiments may include the introduction of a separate cannula (not shown) to the surgical site before a biopsy is performed, such that the biopsy probe 138 is inserted through the cannula to obtain one or more tissue samples. It will be appreciated that any component or procedure that may be introduced or performed through biopsy probe 138 as described herein may also be introduced or performed through such a cannula, among other components or procedures.

In other applications, a diagnostic device (not shown) may be introduced in the same lumen (e.g., the lumen of the piercer tube 24, etc.), prior to, during, and/or after any biopsy being performed. Such a device may include a means for determining the need for tissue removal. Examples of suitable diagnostic devices or diagnostic technologies that may be used may include, but certainly need not be limited to, any of the following: fluid aspiration; molecular assay (e.g., such as a GENESEARCH BLN Assay by Veridex, LLC of Warren, N.J.); a bioconjugate that emits a near-infrared light or other indication when injected (e.g., “tumor painting”); electromagnetic fringe field sensor (e.g., such as by Dune Medical Devices Ltd. of Caesarea Industrial Park, Israel); spectroscopy, such as ambient mass spectroscopy (e.g., desorption electrospray ionization (DESI)); etc., including combinations thereof. Of course, just as a surgical or therapeutic device may be integrated or incorporated with a table 12 or otherwise integrated or incorporated with a system 200, so may a diagnostic device. Similarly, identification and/or authentication of such a diagnostic device may be provided in accordance with routines 300, 400 described herein or otherwise.

In still other embodiments, a diagnostic device is located on a table 12 or near the proximal end of a biopsy device 214, and is not inserted through the lumen of a piercer tube 24. For instance, a diagnostic device may be positioned such that, as tissue is extracted from a patient, it can be immediately analyzed to determine, in substantially real time, if the excised tissue samples are benign or suspicious. Still other suitable locations, positions, and uses of diagnostic devices will be apparent to those of ordinary skill in the art.

Regardless of whether additional devices are provided for surgical, therapeutic, or diagnostic purposes (or for other purposes), it will be appreciated that all or part of such devices may be integrated into the system 200 in a manner similar to that described elsewhere herein with respect to integrating a biopsy system 120 or other ancillary device with system 200. For instance, where an ancillary device that is used for surgical, therapeutic, or diagnostic purposes uses an energy source, integration of the device with the system 200 may be permanent, or may be operable with an energy source that is removable for repair, upgrade, or use in another setting. This integration may be merely physical (e.g., an energy source physically mounted on or in the table 12, etc.), or may be more complete with electrical power, control signals, or even user interface and interaction provided through the table and its control systems (e.g., through the user interface 122 described above). In the present example, surgical energy delivery is available through a connector (not shown) or other attachment to a handpiece (not shown) or disposable energy delivery device (not shown).

To the extent that a device other than a biopsy device 214 is provided for surgical, therapeutic, or diagnostic purposes (or for other purposes), and to the extent that such a device requires physical connection of a cable, fluid conduit, or other component with some integral component of the table 12 or other part of the system 200 (e.g., control to power source 148, data/command communicator 160, etc.), such connections may be proprietary or customized in order to prevent coupling of standard off the shelf cables, fluid conduits, etc. Similarly, where an electronic connection is wireless, a particular encryption or handshake may be used, among other techniques described elsewhere herein to prevent full operability with respect to unauthorized ancillary devices. The system may thus treat such ancillary devices in a manner similar to those described above with respect to routines 300, 400.

In addition, where an additional generator or source of energy, fluid, etc., is required for operability of a non-biopsy device for diagnostic, surgical, or therapeutic use, such a source may be integral with the system 200 similar to vacuum source 144, saline source 16, etc. described elsewhere herein. Such an additional source may include a feature that requires a proprietary or customized connector or communication protocol, etc., as described elsewhere herein, in order for the ancillary device to obtain whatever resource is provided by the source. Non-exhaustive examples of such additional sources may include an RF generator, a laser generator, an ultrasonic generator, a HIFU generator, a microwave generator, an X-ray generator, etc., any of which may be provided as an integral component of a table 12 or otherwise as an integral component of the system 200.

Furthermore, efficiency in performing biopsies, especially in a remote location, may be enhanced by an ability to perform at least a cursory pathology evaluation immediately after acquisition of biopsy samples. To that end, the tube head 86 may be rotated to an offset position aimed at a biopsy container, such as a cylinder sample drum 162. Each sample may be indexed for pneumatic insertion into a respective sample vial 164 with the last received being imaged upon a sample image receptor 166 for analysis by the image processing unit 152. Confirmation of the presence of calcifications may be sufficient to confirm that samples of a lesion of interest have been obtained. It should be appreciated that the single user interface 122 may be in wireless or landline communication with a pathology work station (not shown) for real-time or near real time detailed assessment of the biopsy samples. Of course, a cylinder sample drum 162, sample vial 164, and/or sample image receptor 166 may be integrated into system 200 (e.g., integral with diagnostic table 16) in any suitable fashion, such as in accordance with any of the integrating techniques and structures described herein.

It will also be appreciated that, in some embodiments, in lieu of a tethered remote control, a wireless foot control 158 may be used for actuating the biopsy system 120. To avoid inadvertent actuation of different diagnostic stations 16, various safety interlocks and handshaking routines may be selected. For example, a line-of-sight, range limitation may be imposed upon transmission. As another example, an enabling routine may recall simultaneous sequences of control actuations on both the foot control 148 and the single user interface 122 to confirm a paired arrangement. Third, a serialized code selector may be encoded into both the wireless foot control 158 and an ancillary transceiver 160 of the diagnostic station 16 to enforce a dedicated arrangement. Fourth, a recharging station (not shown) on the diagnostic station 16 may be present. A wireless foot control 158 may be required to be placed into the recharging station for both recharging and for a keyed recognition within a certain time period before use.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art.

For example, it should be appreciated that aspects of the present invention alternatively may be applied to other patient support structures, such as the SENOGRAPHE DS by GENERAL ELECTRIC COMPANY, dba GE Healthcare of the United Kingdom, aspects of which are believed to be described in U.S. Pat. No. 6,611,575, the disclosure of which is hereby incorporated by reference in its entirety. Still other suitable patient support structures will be apparent to those of ordinary skill in the art.

For another example, while an X-ray imaging modality is described in the illustrative versions, it should be appreciated that aspects of the present invention have application to other types of diagnostic imaging currently known or to be developed. By way of example only, suitable alternative imaging techniques may include positive emission tomography (PET), magnetic resonance imaging (MRI), computed tomography (CT), or ultrasound, among others.

As yet another example, interfacing between the biopsy system 120 and the table 12 may reduce the presence of numerous cable and hoses by routing all or substantially all of the necessary conduits and connections through a single mounting that is physically arranged for installation of appropriately verified devices. Electrical and/or physical identification features may configure appropriate electrical, and communication, pneumatic, and fluid supplies to be provided.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A medical apparatus, comprising:
(a) a patient support comprising a breast localization assembly operably configured to compress and localize a breast and at least partially composed of a material transparent in a selected imaging spectrum;
(b) a diagnostic imaging system movable relative to the breast localization assembly for diagnostic imaging of the localized breast;
(c) a biopsy device attaching structure positioned proximate to the breast localization assembly;
(d) a vacuum source and a vacuum port, wherein the vacuum source and vacuum port are integral with the patient support, wherein the vacuum port is configured to be fluidly coupled with a biopsy device engaged with the biopsy device attaching structure; and
(e) a power source, wherein the power source is integral with the patient support, wherein the power source is configured to be operatively coupled with a biopsy device engaged with the biopsy device attaching structure.

2. The medical apparatus of claim 1, further comprising a fluid capture vessel integral with the patient support, wherein the fluid capture vessel is configured to be fluidly coupled with a biopsy device engaged with the biopsy device attaching structure.

3. The medical apparatus of claim 2, wherein the fluid capture vessel is in fluid communication with the vacuum source.

4. The medical apparatus of claim 1, further comprising a supply of liquid, wherein the supply of liquid is integral with the patient support, wherein the supply of liquid is configured to be fluidly coupled with a biopsy device engaged with the biopsy device attaching structure.

5. The medical apparatus of claim 4, further comprising a multi-lumen conduit in communication with the vacuum source and the supply of fluid, wherein the multi-lumen conduit is configured to be fluidly coupled with a biopsy device engaged with the biopsy device attaching structure.

6. The medical apparatus of claim 5, wherein the multi-lumen conduit is formed of a unitary construction.

7. The medical apparatus of claim 1, further comprising a battery charger in communication with the power source.

8. The medical apparatus of claim 7, wherein the battery charger is integral with the patient support.

9. The medical apparatus of claim 8, wherein the battery charger comprises at least one battery receptacle.

10. The medical apparatus of claim 1, further comprising a user interface integral with the patient support, wherein the user interface is operable to control at least two of the patient support, the diagnostic imaging system, or a biopsy device coupled with the biopsy device attaching structure.

11. The medical apparatus of claim 10, further comprising a communicator integral with the patient support, wherein the communicator is operable to communicate one or both of data or commands between the user interface and a biopsy device coupled with the biopsy device attaching structure.

12. The medical apparatus of claim 11, wherein the communicator is operable to communicate one or both of data or commands wirelessly.

13. The medical apparatus of claim 1, further comprising a source of pressurized air integral with the patient support, wherein the source of pressurized air is configured to be fluidly coupled with a biopsy device engaged with the biopsy device attaching structure.

14. The medical apparatus of claim 1, further comprising an ancillary device authentication feature operable to evaluate the authenticity of an ancillary device coupled with the medical apparatus, wherein the ancillary device authentication feature is further operable to regulate operability of at least one of the patient support, the diagnostic imaging system, an ancillary device coupled with the medical apparatus, the vacuum, source, or the power source.

15. The medical apparatus of claim 14, wherein the ancillary device authentication feature is integral with the patient support.

16. An integrated medical system, the system comprising:
(a) a patient support comprising a breast localization assembly operably configured to compress and localize a breast;
(b) a diagnostic imaging system for diagnostic imaging of the localized breast;
(c) a biopsy device attaching structure positioned proximate to the breast localization assembly;
(d) a vacuum source and a vacuum port, wherein the vacuum source and vacuum port are integral with the patient support, wherein the vacuum port is configured to be fluidly coupled with a biopsy device engaged with the biopsy device attaching structure;
(e) a fluid vessel integral with the patient support, wherein the fluid vessel is configured to be fluidly coupled with a biopsy device engaged with the biopsy device attaching structure; and
(f) a power source, wherein the power source is integral with the patient support, wherein the power source is configured to be operatively coupled with a biopsy device engaged with the biopsy device attaching structure.

17. The integrated medical system of claim 16, wherein the fluid vessel comprises a fluid capture vessel in fluid communication with the vacuum source.

18. The integrated medical system of claim 16, wherein the fluid vessel comprises a source of saline.

19. The integrated medical system of claim 16, wherein the fluid vessel comprises a pressurized fluid.

20. A medical system, comprising:
(a) a patient support comprising a breast localization assembly operably configured to compress and localize a breast;
(b) a diagnostic imaging system for diagnostic imaging of the localized breast;
(c) a biopsy device attaching structure positioned proximate to the breast localization assembly;
(d) a vacuum source and a vacuum port, wherein the vacuum source and vacuum port are integral with the patient support, wherein the vacuum port is configured to be fluidly coupled with a biopsy device engaged with the biopsy device attaching structure; and
(e) a power source, wherein the power source is integral with the patient support, wherein the power source is configured to be operatively coupled with a biopsy device engaged with the biopsy device attaching structure; and
(f) an integrated user interface in communication with the power source, wherein the user interface is operable to control each of the patient support, the diagnostic imaging system, a biopsy device coupled with the biopsy device attaching structure, and the vacuum source.
